(19) United States
(12) Patent Application Publication
(10) Pub. No.: US 2006/0020171 A1
(43) Pub. Date: Jan. 26, 2006

(54) Intubation and imaging device and system

(76) Inventor: Mark G. Gilreath, Cumming, GA (US)

Correspondence Address: PEARL COHEN ZEDEK, LLP, 10 ROCKEFELLER PLAZA, SUITE 1001, NEW YORK, NY, 10020, US

(21) Appl. No.: 10/531,692

(22) PCT Filed: Oct. 21, 2003
(86) PCT No.: PCT/IL03/00853

§ 371 Date: Apr. 18, 2005

Related U.S. Application Data
(60) Provisional application No. 60/419,558, filed on Oct. 21, 2002.

(51) Int. Cl. A61B 1/267; A61B 1/06

(52) U.S. Cl. 600/188; 600/109; 600/179; 600/160

(57) ABSTRACT

An intubation tool may include an imaging unit. The imaging unit may be, for example, self contained, autonomous and/or single use. Images may be transmitted to, for example, an external receiving system.

### FIELD OF THE INVENTION

The present invention relates to the field of medical devices, more specifically to an endotracheal intubation tool having integrated visualization and to a method for its use.

### BACKGROUND OF THE INVENTION

Endotracheal intubation is a common medical procedure often directed at opening a closed larynx by inserting a laryngoscope through the larynx followed by the insertion of an endotracheal tube, which enables air supply to the patient. Endotracheal intubation is a typically life saving procedure performed in emergency cases. Thus, the ability to intubate a patient rapidly is highly important.

In many patients, intubation may be particularly difficult to perform due to morphological anomalies, such as a large tongue, excessive soft tissue, or tracheal displacement. These morphological anomalies may make it difficult to visualize the posterior pharyngeal area, larynx and cords, and may cause difficulties in intubation. In medical emergency situations, an attempt to intubate such persons may be difficult, time consuming, and may meet with failure. Other situations may make intubation and/or the associated viewing difficult.

To overcome this problem intubation devices have been developed which include, for example, illumination and visualizing components for illuminating and visualizing the pharynx, larynx, trachea and associated structures, during intubation.

The illumination and visualization is typically performed by using fiber optics both for illuminating and for viewing. Optical fibers are typically connected by, for example, wires or a bus to a power supply source and to an illumination source, both typically located outside a patient's body. Optical fibers and wires may take up space within an intubation tool, and also may restrict the free movement and ability to maneuver the intubation tool. Further, in some devices an external power source may be required.

### SUMMARY

Some embodiments of the present invention include endotracheal intubation tools such as laryngoscopes, and may include for example an imaging unit, which allows typically continuous in vivo visualization during insertion, and possibly during use, of the laryngoscope. Endotracheal intubation may be a life saving procedure performed in emergency situations, such as in the case of an obstructed airway. Thus, the procedure, according to embodiments of the invention may be performed in the field, outside of a medical center or hospital. Embodiments of the present invention may have the benefit of performing endotracheal intubation procedures while enabling the viewing of inner cavities, lumens, organs, etc. of the patient in-vivo, while in the field. Other embodiments may allow for treatment in other settings, such as a hospital.

Embodiments of the present invention provide a device, system and method allowing for effective intubation through the use of an intubation device or tool with an improved imaging system..

In one embodiment the invention provides an intubation tool that includes a handle; a blade; and at least one imaging unit. The handle and blade are typically releasably interlockable or attachable to each other. Typically, there is a passageway or channel through the handle and blade. The tool may include, for example, a bivalve element, typically used for forming the passage. The imaging unit typically includes an image sensor and an illumination source. According to one embodiment both image sensor and illumination source are situated behind an optical window. According to some embodiments the intubation tool may include a transmitter, typically for transmitting signals, such as image data. The intubation tool may also include a power source for powering components of the imaging unit.

According to one embodiment the intubation tool is connected to the blade, typically to a distal end of the blade, although other positions are possible.

A system for intubation, according to embodiments of the invention may include an intubation tool, which includes a handle; a blade; at least one imaging unit; and a transmitter; and a receiving unit for receiving signals transmitted from the transmitter. The system may include a processing unit for, for example, processing received signals and a display. According to one embodiment image data is displayed on the display.

In some embodiments, an intubation tool or device may have visualization capabilities that may take up less space within the tool or device, and may not restrict the free movement and ability to maneuver the intubation tool. In some embodiments, the imaging unit may be, for example, self contained, autonomous and/or single use.

### BRIEF DESCRIPTION OF THE DRAWINGS

The present invention will be understood and appreciated more fully from the following detailed description taken in conjunction with the drawings in which:

FIG. 1 is a schematic illustration of an intubation system in accordance with an embodiment of the invention;

FIG. 2 is a schematic illustration of an intubation system in accordance with another embodiment of the invention; and

FIGS. 3A-3B are schematic illustrations of an imaging unit in accordance with embodiments of the invention.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

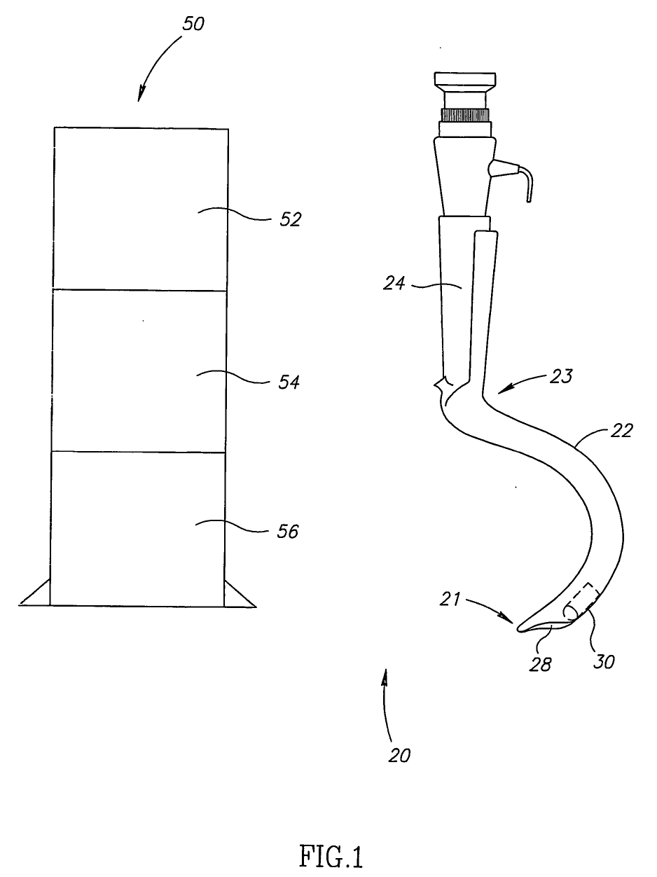

Reference is now made to FIG. 1, which is a schematic illustration of an intubation system 10 in accordance with an embodiment of the invention. The intubation system 10 typically includes a laryngoscope 20, an imaging unit 30 attached to or included as part of laryngoscope 20 and a receiving unit 50. receiving unit 50, which, according to one embodiment includes a receiver 52, a processor 54 and a screen or display 56, receives signals, for example image data, from imaging unit 30 and processes the signals, for example, to form an image. According to one embodiment imaging unit 30 is located at the distal end 21 of the laryngoscope 20. In alternate embodiments imaging unit 30 may be located at any other suitable site on the laryngoscope 20.

In some embodiments, the imaging unit may be, for example, self contained, autonomous and/or single use. For example, the imaging unit may be a self contained or encapsulated unit which can operate without external power and without a tether or cable required to send images to a viewer. In other embodiments, power may be provided by, for example, an external source, but without a wire or cable; for example, via magnetic waves.

In one embodiment laryngoscope 20 is a two piece laryngoscope that includes a blade 22 and a handle 24. According to one embodiment handle 24 is attached to the proximal end 23 of blade 22. In other embodiments, other devices or tools may be used having other configurations; for example a blade and handle may not be separate pieces.

In one embodiment blade 22 is a curved structure attached to the handle 24 at a convenient angle to be place over a patient's tongue, for example, as described in U.S. Pat. No. 4,982,729 to Wu, incorporated herein by reference in its entirety. For example, in one embodiment the axis of the handle and the axis of the blade are at an angle of about 100 degrees to 120 degrees. Other suitable angles may be used. In such an embodiment, the laryngoscope may include, for example, an integral handle 24 and blade 22. The blade 22 may optionally include a bivalve element 28 that is, for example, releasably attachable to the blade 22 and/or the handle 24 to form a passageway for, for example, threading an endotracheal tube to the distal end of the blade 22.

The handle 24 may be attached to the blade 22 at an angle so that the blade 22 may enter the upper oral cavity with minimal maneuvering of the patient's head and neck. According to one embodiment the blade 22 may be laterally curved to form a groove running the length of the blade 22 and opening toward the convex portion of the blade 22. The laryngoscope may include a bivalve element (e.g., 28) shaped to correspond with the shape of the blade 22, including a longitudinal groove opening toward the concave side of the curved portion. In such a case the groove in the blade 22 may connect with a groove in the bivalve element when they are positioned together to form a passageway from the handle 24 to the distal end 21 of the blade 22. The bivalve element 28 may be releasably attachable to the proximal end 23 of the blade 22 as well as being releasably attachable to the handle 24. The passageway formed between the blade 22 and the bivalve element 28 may be large enough in diameter to hold, for example, an endotracheal tube.

According to one embodiment the two parts of the laryngoscope 20 may be assembled, for example, in bivalve fashion before insertion into the patient's mouth. The interconnected bivalve elements may be disconnected within the patient's throat after an endotracheal tube has been inserted into the patient's trachea and intubation has been effected, typically so that the laryngoscope can be removed from the patient in two pieces leaving the endotracheal tube in place. In one embodiment the blade may include a substantially straight section attached to the handle, with an arced mid-portion, and a straight or substantially straight distal portion. Other blades may be used, having other components and configurations, having other functionalities and uses, and in alternate embodiments a blade need not be used. Other tools may be used.

Laryngoscope 20 may be a single use laryngoscope. Alternatively, the handle 24 may be a multi use piece and the blade 22 may be a single use piece. Alternatively, laryngoscope 20 may be a multi use laryngoscope.

In other embodiments laryngoscope 20 may be of any suitable kind including, for example, a multi piece laryngoscope, a laryngoscope including a tube for air passage or for insertion of tools, a laryngoscope having a straight blade, a curved blade, various shapes of handles, or other equipment, etc. The device, system and method of the present invention may be used with suitable endotracheal tools other than a laryngoscope 20.

Imaging unit 30 typically captures in-vivo images. According to some embodiments other in vivo sensing units (e.g., pressure sensor, blood detector, temperature sensor etc.) may be included in an imaging unit, which may facilitate correct and easy insertion. According to one embodiment imaging unit 30 may be a single use unit that may be attached to a single use laryngoscope or to the single use blade 22 of a laryngoscope, or to another structure having a single use blade. By utilizing single use parts the need to sterilize the device in between uses may be avoided. Alternatively, a single use imaging unit 30 may be attached to a multi use laryngoscope such that only imaging unit 30 may be changed between uses. Alternatively, a multi use imaging unit 30 may be attached to a multi use laryngoscope.

The attachment of imaging unit 30 to laryngoscope 20 may be achieved for example, by gluing, soldering, clamping or other mechanical attachment or by other suitable methods. Imaging unit 30, or a shell or covering for imaging unit 30, may be integral with laryngoscope 20 or another tool Imaging unit 30 may be a wireless imaging unit. A wireless imaging unit may enable comfortable and flexible use of laryngoscope 20. Furthermore, the wireless imaging unit may enable free maneuvering of the laryngoscope during the procedure while simultaneously viewing the images captured by wireless imaging unit 30. Free maneuvering is desirable, especially in use with patients having morphological anomalies.

In one embodiment wireless imaging unit 30 may be a one piece unit, that may be, for example autonomous. One piece unit 30 may be located near or at the distal end 21 of blade 22 to enable imaging of a patient at the site of treatment or operation. In alternate embodiments imaging unit 30 may be located at any other point on the system 10, or at another place in the patient suitable for enabling in-vivo imaging.

In alternate embodiments, imaging unit 30 may include multiple parts which may be, for example, separately located along the overall tool, or may, for example, be a wired imaging unit connected by, for example, wires or a bus to a power supply system and/or to receiving unit 50.

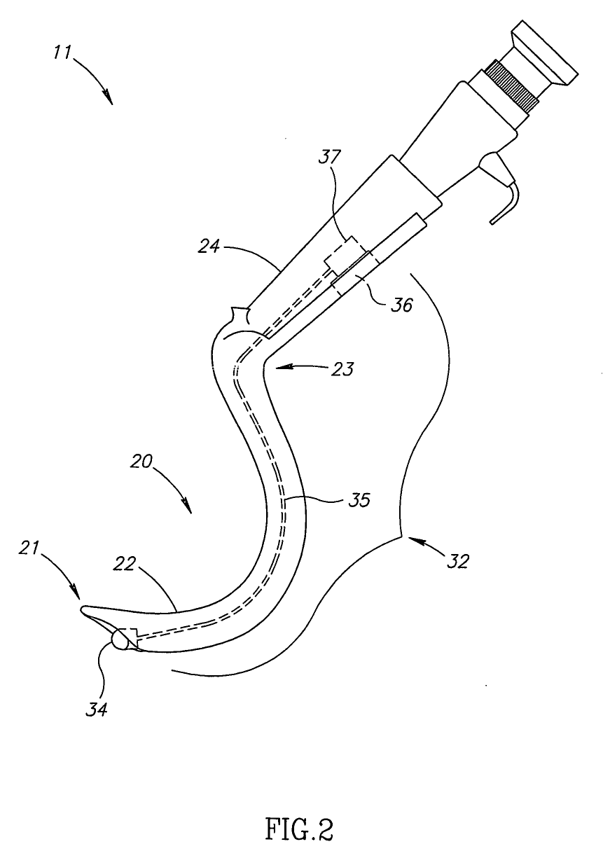

The receiving unit 50 may receive signals from one piece imaging unit 30 or from multi piece imaging unit (e.g., 32 in FIG. 2) and may process the signals by processor 54 to an output (e.g., image output) displayed on a screen 56. The signals may be received by receiver 52 through, for example, wireless communication between one piece unit 30 or multi piece unit 32 and receiver 52. In one embodiment, the receiving unit 50 may be part of a portable computer. This may enable, for example, viewing of in-vivo images in field conditions without having wires complicate the procedure.

Processor 54 typically is or includes a computer that processes the received signals to create an image. Screen 56 is typically a screen such as a computer monitor that enables presenting a view of the images. Receiver 52 may be connected to processor 54 by, for example, wires or a bus. Alternatively, receiver 52 may be incorporated in processor 54 or in a unit incorporating processor 54. Display unit 50 may have other structures or components.

A reception and display system used with the system and method of the present invention may (with possible modifications) be similar to those, or may use components and methods similar to those, described in U.S. Pat. No. 5,604,531 to Iddan et al. and/or in International Application publication number WO 01/65995 entitled “A Device And System For In Vivo Imaging”, published on 13 Sep., 2001, each of which are assigned to the common assignee of the present application, and each of which are incorporated herein by reference in their entirety.

In use, receiving unit 50 is typically located outside the patient body while the one piece imaging unit 30 is typically located in-vivo. In the case of a multi piece imaging unit, a first imaging piece may be located in-vivo and a second transmitting and/or power supplying piece may be located outside the patient body, for example, in the handle, or attached to the patient.

Reference is now made to FIG. 2, which is a schematic illustration of an intubation system 11 in accordance with an embodiment of the invention where the imaging unit may be a multi piece unit. Laryngoscope 20 includes a multi piece unit 32 According to one embodiment multi piece unit 32 includes an imaging unit 34, a transmitter 37 and a power supplying unit 36 (e.g., batteries, a power receiving unit, or another suitable power supply). The communication between imaging unit 34 and transmitter 37 and/or power supplying unit 36 may be conducted via, for example, a wire or set of wires 35 progressing, for-example, along the internal side of blade 22 and handle 24. The wire(s) 35, the imaging unit 34, and transmitter 37 and/or power supply unit 36 may be located differently. Imaging unit 34 is typically located near or at the distal end 21 of blade 22 to enable in-vivo imaging, for example, at the site of operation or treatment. It may be attached, for example, at the inner side of the distal end 21 of blade 22. The attachment of imaging unit 34, wire(s) 35 and transmitter 37 and power supplying unit 36 to laryngoscope 20 may be achieved for example, by gluing, soldering, clamping or other mechanical methods, or by other methods; various components may be integral with laryngoscope 20 or other parts of the tool. According to some embodiments the multi piece unit 32 may enable further miniaturizing of the piece attached to blade 22.

Multi piece imaging unit 32 may transmit, typically through transmitter 37 or otherwise send signals to receiving unit 50 (FIG. 1) where the signals are received by receiver 52 and typically processed by processor 54 to produce an image that can be viewed on screen 56.

In alternate embodiments multi piece unit 32 may be an imaging unit connected by, for example, wires or a bus to a power supply system and/or to receiving unit 50.

The operation of laryngoscope 20 may be assisted by viewing in vivo images captured by one piece unit 30 or multi piece unit 32. Organs or structures at the site of the operation may be imaged and viewed simultaneously via receiving unit 50, in real time, during operation of laryngoscope 20.

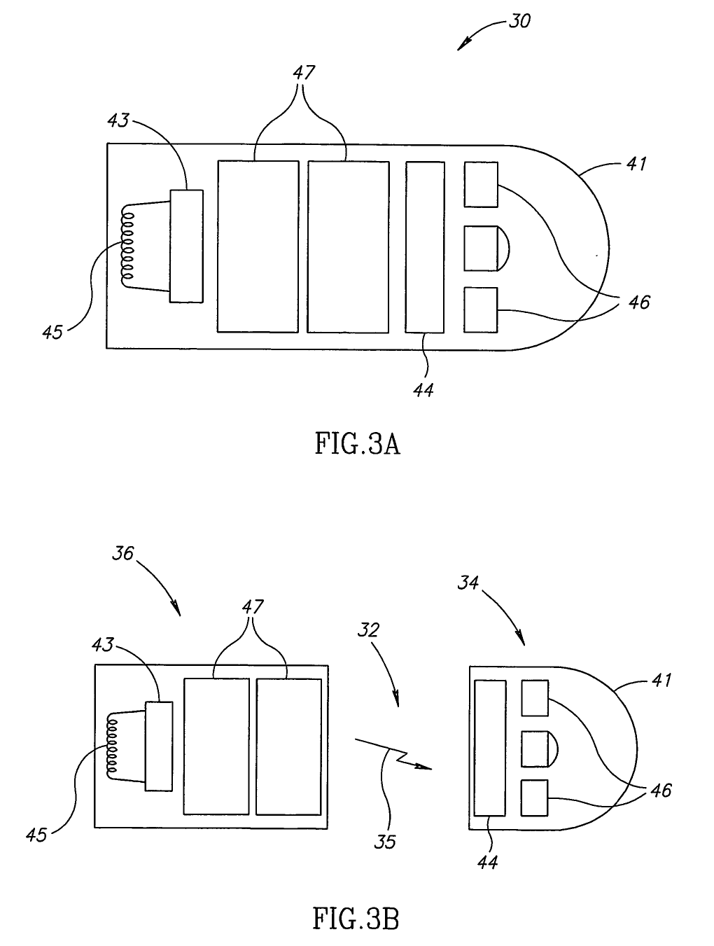

Reference in now made to FIG. 3A, which is an illustration of an in-vivo imaging unit according to an embodiment of the invention. One piece unit 30 typically includes an image sensor 44 (such as, for example, a CMOS or CCD camera, or another imager) and an illumination source 46 (such as, for example, white LEDs, or another illumination source). The components of one piece unit 30 may be used in other embodiments discussed herein. Optionally, image sensor 44 and illumination source 46 may both be situated behind a dome or other shaped optical window 41. According to one embodiment the one piece unit 30 further includes a transmitter 43, optionally with an antenna 45, that transmits signals from the image sensor 44 to the receiving unit 50, either wirelessly, for example by using radio waves (e.g., “RF”), or through a wire connection. The wireless transmission of signals from the image sensor 44 to receiver 52 may be effected using, for example, various digital or analog modulation techniques. For example, transmission of a digital image over a radio channel may use an FSK (Frequency Shift Keying) modulation technique. Other transmission techniques may be used.

One piece unit 30 may include a power supply 47 (such as, for example, one or more silver oxide batteries, rechargeable batteries, etc.), for supplying the electric power required for the operation of the one piece unit 30. Other imaging units, and other power supplies, may be used.

Image sensor 44 may be, for example, a CCD or an active or passive CMOS imaging chip and may generate digital or analog signals. In one embodiment, image sensor 44 is a single chip imager such as or similar to the CMOS image sensor (“Camera on Chip”) designed by Photobit Inc. of Calif., USA, with integrated active pixel and post pixel circuitry. Typically, the one piece unit 30 also includes an optical system (such as lenses, mirrors etc.).

In some embodiments, the imaging unit and its use, and other imaging systems described herein, may be similar to embodiments disclosed in U.S. Pat. No. 5,604,531 and/or International Application publication number WO 01/65995. A device, system and method of embodiments of the present invention may be used with other imagers, image processing systems, and imaging systems, having different structures and components.

It will be appreciated that a plurality of imaging cameras may be used in the device, system and method of the invention. Each imaging camera may include its own optical system and either one or more illumination sources, in accordance with specific requirements of the device or system.

Typically both image sensor 44 and illumination sources 46 are low power components such that they may be powered by one or more batteries and thus may not require wire connection to an external power supply system. Other imaging or lighting systems may be used, and power by wire may be used.

Reference is now made to FIG. 3B, which is an illustration of a multi piece unit 32 according to one embodiment of the invention. Multi piece unit 32 includes, for example, a first, imaging piece 34 situated, for example, at the distal end of blade 22. Multi piece unit 32 may include a second piece 36 including, for example, transmitting and power supply capabilities and situated at a remote location, for example, in the handle 24. According to other embodiments transmitting capabilities and power supply capabilities may be included in separate units. Other connection points for the first and second piece are possible. The first and second pieces 34 and 36 may be connected through, for example, wire(s) 35. In alternative embodiments piece 34 and piece 36 communicate completely or partially wirelessly, such as by microwave, infrared (IR) or radio waves (RF); other wireless communication methods may be used. Furthermore, other communications methods, such as fiber optic, may be used.

The first, imaging, piece 34 typically includes imager 44 (for example a CMOS camera) and at least one illumination source 46 (for example white LEDs) both situated behind a typically dome or other shaped optical window 41. The second piece 36 typically provides transmitting and power supply functionality, and includes a power supply 47 (e.g. one or more batteries), a transmitter 43 and an antenna 45 that transmits signals from the imager 44 to an external receiving system (for example display unit 50 in FIG. 1). Such an arrangement may allow miniaturizing of the first piece of imaging unit 32 located at the distal end of blade 22. The components may be divided between more than two pieces.

In another embodiment the power supply 47, which may convey energy to the imaging unit 30 either wirelessly or through a wired connection, may be situated outside a patient's body.

It will be appreciated by persons skilled in the art that the present invention is not limited to what has been particularly shown and described hereinabove. Alternate embodiments are contemplated which fall within the scope of the invention.

1. An intubation tool comprising:
a handle;
a blade; and
at least one imaging unit,
said handle and blade being releasably interlockable with each other.

2. The intubation tool according to claim 1 having a passageway through the handle and the blade.

3. The intubation tool according to claim 1 wherein the imaging unit comprises an image sensor and an illumination source.

4. The intubation tool according to claim 3 wherein the image sensor and illumination source are located behind an optical window.

5. The intubation tool according to claim 1 comprising a transmitter.

6. The intubation tool according to claim 5 wherein the transmitter is wireless.

7. The intubation tool according to claim 1 comprising a power source.

8. The intubation tool according to claim 1 wherein the imaging unit is connected to the blade.

9. The intubation tool according to claim 1 wherein the imaging unit is positioned at a distal end of the blade.

10. The intubation tool according to claim 2 comprising a bivalve element, said element being releasably attachable to the handle and to the blade.

11. A system for intubation, the system comprising an intubation tool, said tool comprising:
a handle;
a blade;
at least one imaging unit; and
a transmitter;
said handle and blade being releasably interlockable with each other; and
a receiving unit for receiving signals transmitted from the transmitter.

12. The system according to claim 11 wherein the transmitter is wireless.

13. The system according to claim 11 comprising a processing unit for processing received signals.

14. The system according to claim 11 wherein the signals include image data.

15. The system according to claim 11 comprising a display.

16. The system according to claim 11 wherein the intubation tool includes single use components.

17. A device comprising:
a handle releaseably connected to a blade; and
an imager.

18. The device according to claim 17 comprising a channel through the blade.

19. The device according to claim 17 comprising a power source.

20. The device according to claim 17 comprising a radio transmitter.
